(19) United States
(12) Patent Application Publication
(10) Pub. No.: US 2012/0041533 A1
(43) Pub. Date: Feb. 16, 2012

(54) STENT DELIVERY DEVICE

(75) Inventors: William C. Bertolino, Framingham, MN (US); Paul Aquilino, South Walpole, MA (US); Chris Benning, Lowell, MA (US)

(73) Assignee: BOSTON SCIENTIFIC SCIMED, INC., Maple Grove, MN (US)

(21) Appl. No.: 13/206,820

(22) Filed: Aug. 10, 2011

Related U.S. Application Data
(60) Provisional application No. 61/372,302, filed on Aug. 10, 2010.

(51) Int. Cl. A61F 2/82

(52) U.S. Cl. 623/1.11

(57) ABSTRACT

Various methods and devices are described for imaging a body lumen during delivery and deployment of a medical device. In one example, a delivery device includes at least one sheath, a stent, an inner tubular member and at least one imaging device to allow visualization of the stent prior, during and after deployment without the use of an endoscope. The inner tubular member includes an articulating portion with the imaging device integrally formed and embedded therein.

This application claims the benefit of U.S. Provisional Application No. 61/372,302, entitled “ARTICULATING STENT DELIVERY DEVICE AND STENT GUIDEWIRE DELIVERY DEVICE WITH IMAGING,” by William Bertilino, Paul Aquilino, and Chris Benning, and filed on Aug. 10, 2010, the entire contents of which being incorporated herein by reference.

### TECHNICAL FIELD

The disclosure relates to medical devices and, in particular, to medical delivery devices for imaging within a body lumen.

### BACKGROUND

Stents and stent delivery assemblies are utilized in a number of medical procedures and situations and, as such, their structure and function are well known. A stent is a generally cylindrical prosthesis that is introduced via a catheter into a lumen of a body cavity or vessel. The stent is introduced into the cavity or vessel with a generally reduced diameter and then is expanded to the diameter of the cavity or vessel. In its expanded configuration, the stent supports and reinforces the cavity/vessel walls while maintaining the cavity/vessel in an open, unobstructed condition.

A stent delivery catheter is typically delivered over a guidewire. A guidewire is very flexible and has a smaller diameter than a stent delivery catheter, and therefore is inserted into the body cavity or vessel of interest first, over and along which a stent delivery catheter can follow.

Typically, when delivering a stent into a body cavity of interest, a guidewire is introduced into the body cavity through a working lumen defined in an endoscope. An example of an endoscope used in lumens is described in U.S. Pat. No. 7,591,785, the entire content of which being incorporated herein by reference. A physician advances an endoscope and the guidewire removably received therethrough into the body cavity of interest while observing an image received from the distal end of the endoscope. Once the distal end of the guidewire reaches the position of interest, as observed by the endoscope, the endoscope is withdrawn, leaving the guidewire in place. Thereafter, a stent delivery catheter is passed over the guidewire and the stent is deployed. To observe and ensure proper deployment of the stent, the endoscope is sometimes passed along the side of the stent during deployment. In addition, for example, when applying a stent in a blood vessel, fluoroscopy (x-ray imaging of a moving object) is often used to ensure proper placement and deployment of the stent, as well known in the art.

### SUMMARY

In one example, the disclosure is directed to a delivery device comprising at least one sheath removably covering a stent therein, said at least one sheath comprising a distal end, a proximal end, an outer surface and a working channel extending between said distal end and said proximal end, said working channel defining an inner wall. The stent defines a stent lumen, said stent extending in a compressed state within said working channel. The delivery device further comprises an inner tubular member slidably disposed within said stent lumen, said inner tubular member comprising an elongated inner shaft with a distal articulating portion extending therefrom. The delivery device further comprises at least one imaging device integrally formed in said distal articulating portion.

In another example, the disclosure is directed to a method for intraluminally positioning a prosthesis comprising providing a delivery device comprising at least one sheath removably covering a prosthesis therein, said at least one sheath comprising a distal end, a proximal end, an outer surface and a working channel extending between said distal end and said proximal end, said working channel defining an inner wall, said prosthesis extending in a compressed state within said working channel, an inner tubular member slidably disposed within said prosthesis, said inner tubular member comprises an elongated inner shaft with a distal articulating portion extending therefrom, and at least one imaging device integrally formed in said distal articulating position; activating said at least one imaging device to provide images during positioning of said prosthesis; positioning said delivery device within a body lumen; and slidably retracting said at least one sheath relative to the inner tubular member to uncover said prosthesis and allow said prosthesis to radially expand against a wall of body lumen, wherein said articulating position is bent back upon itself to allow said at least one imaging device to be positioned for visual inspection of deployment of the prosthesis while slidably retracting said at least one sheath.

The details of one or more aspects of the disclosure are set forth in the accompanying drawings and the description below. Other features, objects, and advantages will be apparent from the description and drawings, and from the claims.

### BRIEF DESCRIPTION OF DRAWINGS

FIG. 1 is a schematic view of one example delivery system in accordance with various techniques described in this disclosure.

FIG. 2 is a schematic view of the example delivery system of FIG. 1 showing the articulating member bending 180 degrees from the original position.

FIG. 3 is a schematic view of another example delivery system in accordance with various techniques of this disclosure.

FIG. 4 is a schematic view of another example delivery system in accordance with various techniques of this disclosure.

Corresponding reference characters indicate corresponding parts throughout the several views of the drawings.

### DETAILED DESCRIPTION

Endoscopes are commonly used to deliver stents into a body cavity. When delivering a stent in a body cavity of interest, a guidewire is introduced into the body cavity through a working lumen defined in an endoscope. An endoscope, however, has a diameter that is relatively large with respect to the body cavity or body lumen of interest. Thus, the use of an endoscope to deliver a guidewire (and hence a stent delivery catheter) becomes more difficult in some applications. For example, esophageal, gastrointestinal (GI), and pulmonary stents are fairly large, thereby requiring a larger delivery system. Therefore, positioning an endoscope along the side of a stent to observe its proper deployment requires an even larger space, which is not always available. Still further, use of fluoroscopy to confirm proper positioning of a guidewire and/or a stent is a relatively cumbersome procedure and requires additional safety mechanisms for the patients as well as the doctors and their assistants.

As such, a need exists for a vision system that is integral with the stent delivery system to provide a smaller device that deploys and provides vision, as well as preventing introduction and reintroduction of multiple devices and steps. Additionally, a need exists for a stent delivery system having imaging capabilities to allow visualization of stent prior, during and after deployment without the use of an endoscope.

In general, this disclosure describes delivery devices for delivering medical device, e.g., stents, that may include an articulating tubular member extending through the lumen of the stent and one or more imaging devices and illumination devices, e.g., integrally formed and embedded into the articulating member. The articulating member may articulate using various techniques including using, for example, pull wires, shape memory material, and electroactive polymers. The delivery devices described in this disclosure include an enlarged central lumen to permit passage of the imaging device(s).

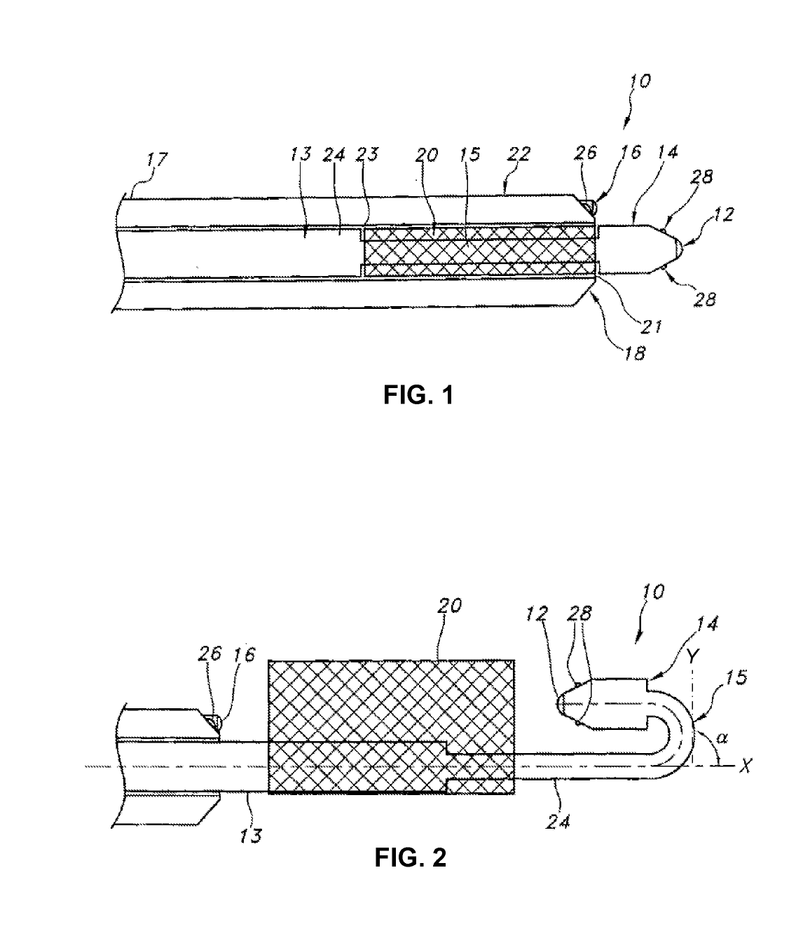

FIGS. 1 and 2 depict schematic views of one example delivery system in accordance with various techniques described in this disclosure. In FIGS. 1 and 2, delivery device 10 includes imaging devices 12, 16 that may, for example, be integrally formed and embedded into the delivery device. FIG. 1 shows delivery device 10 including articulating tubular member 24 extending within outer sheath 22. Outer sheath 22 includes proximal end 17, distal end 18, and working channel 21, which defines inner wall 23, extending therebetween. Outer sheath 22 may have a hollow tubular shaft that removably covers stent 20 and retains stent 20 in a compressed position until deployment.

Articulating tubular member 24 extends within the lumen of stent 20, and stent 20 slidably extends between the articulating tubular member 24 and the outer sheath 22. Articulating tubular member 24 may be a tubular shaft, e.g., solid or hollow, and may have a guidewire extending therethrough (not shown). In some examples, articulating tubular member 24 may be a continuous elongated shaft extending between distal tip 14 and a proximal end (not shown). In one example, articulating tubular member 24 includes proximal portion 13, distal portion 15, and distal tip 14 extending distally from distal portion 15. In some example configurations, distal portion 15 may have a smaller diameter than proximal portion 13, as shown in FIGS. 1 and 2.

Delivery device 10 may include one or more imaging devices, e.g., one, two, three, four, or more. FIG. 1 depicts an example delivery device 10 including two imaging devices, namely first imaging device 12 and second imaging device 16. First imaging device 12 may be located within distal tip 14 of delivery device 10. For example, first imaging device 12 may be integrally formed from and embedded into distal tip 14 of delivery device 10. First imaging device 12 may allow for evaluation of the anatomy prior to stent deployment.

Second imaging device 16 may be located at distal end 18 of outer sheath 22. In one example, second imaging device 16 may be integrally formed from and embedded into outer sheath 22 of delivery device 10. Second imaging device 16 may allow for observation of a proximal end of stent 20 during stent release and provide a proximal view of the stent during deployment, as shown in FIG. 2. In other example configurations, second imaging device 16 may be located on distal tip 14 and/or anywhere along proximal portion 13 of articulating tubular member 24.

The imaging devices described in this disclosure, e.g., imaging devices 12 and 16 of FIGS. 1 and 2, may include, but are not limited to, cameras such as an imaging chip and a lens, e.g., omnivision image chip with about 77 kpixels. Additionally, the camera may include one or more imaging fiber bundles, where fiber optics are used instead of a camera, e.g., the SpyGlass® Imaging System available from Boston Scientific. The images from the cameras may be sent as imaging signals to an external display device via wired or wireless signal transmission techniques. Additionally, in some examples, the camera can be a rotation camera such that it moves/rotates to different positions/angles within a socket. Further, the cameras may utilize a variety of different lenses, e.g., fixed lenses, focusable lenses, wide angle, macro/micro lens and the like.

Referring now to FIG. 2, after stent 20 has been released, first imaging device 12 may be used to confirm stent placement and re-inspect the anatomy. Using various techniques of this disclosure, distal portion 15 articulates about the axis X of the delivery system. Articulating, as used herein, refers to bending, flexing, movement of a member or portion into a non-linear position, curving, arcing, and the like. In some examples, distal tip 14 and first imaging device 12 articulate, e.g., bend backwards, such that first imaging device 12 points in a direction that is substantially opposite (about 180°) to the direction that first imaging device 12 points in an unarticulated position (FIG. 1), as shown in FIG. 2. In FIG. 2, angle α defines an angle between the axis X of the delivery system and axis Y, which is an axis tangential to a point on articulating tubular member 24. In order for first imaging device 12 to point in a direction that is substantially opposite (about 180°) to the direction that first imaging device 12 points in an unarticulated position (FIG. 1), angle α is about 90°. Articulating member 24 may articulate at smaller angles. For example, first imaging device 12 may point in a direction that is substantially perpendicular (about 90°) to the direction that first imaging device 12 points in an unarticulated position (FIG. 1)(not depicted). In such an example, angle α is much less than 90°. Other angles are within the scope of this disclosure.

Articulating member 24 may articulate in various directions in a rotation about the axis X to examine the deployed stent. Distal portion 15 may be made from a flexible geometry and/or flexible material which allows articulation up to about 180 degrees, such as segmented sections or joints, or flexible material such as Nitinol, or a flexible polymer or elastomer.

As indicated above, articulating member 24 may articulate by way of pull wires, shape memory material, and electroactive polymers, for example. For example, articulating member 24 of delivery device 10 of FIGS. 1 and 2 may formed from shape memory material, which have unique characteristics. The unique characteristic of such material is the materials thermally triggered shape memories, which allows the material to regain a memorized shape when warmed to a selected temperature, e.g., human body temperature. The two different shapes are possible because of the two different crystalline structures which exist in such materials at different temperatures.

Referring to FIG. 2, articulating member 24 may be formed of a shape memory material having a first shape in a first state. In particular, FIG. 2 depicts articulating member 24 having a first shape, i.e., articulated, when in a first state, e.g., when exposed to body temperature. Articulating member 24 may then be bent, compressed, or otherwise forced into a second shape when in a second state. In particular, FIG. 1 depicts articulating member 24 having a second shape when constrained, e.g., by outer sheath 22, when in a second state, e.g., when exposed to temperatures cooler than body temperature. As delivery device 10 is advanced into a body lumen and articulating member 24 is exposed to body temperature, articulating member 24 begins attempting to regain its memorized articulated shape. Outer sheath 22, however, prevents articulating member 24 from articulating. Once at the stent deployment site, outer sheath 22 is retracted and articulating member 24 regains its memorized shape.

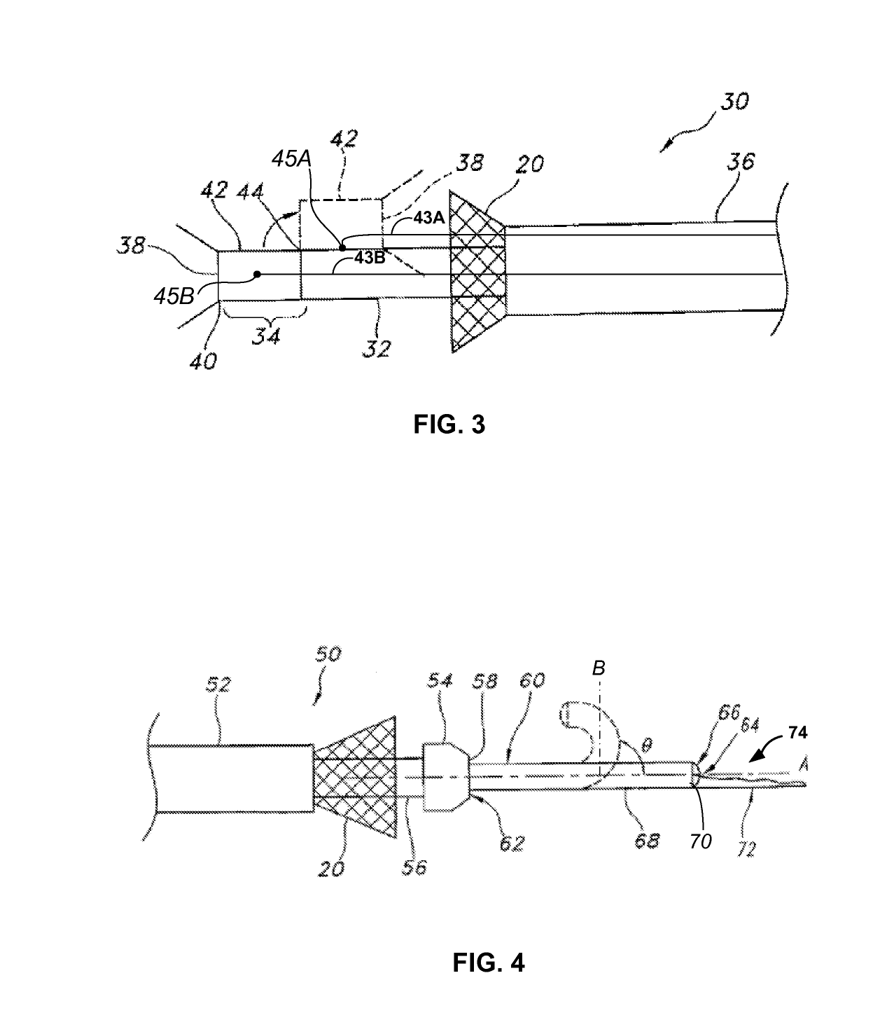

Although FIGS. 1 and 2 were described above with respect to shape memory material, the disclosure is not so limited. Rather, in some examples, a clinician may articulate articulating member 24 by way of pull wires. One example configuration using pull wires is described below with respect to FIG. 3 and, for purposes of conciseness, will not be described again.

Within close proximity to imaging devices 12, 16, delivery device 10 may include illumination devices 28, 26, respectively, to provide illumination within the lumen. The illumination devices 28, 26 may be located on either side of the imaging devices 12, 16, respectfully. A portion of the stent may light up to illuminate the stent rather than having the camera attached to the delivery device.

Illumination devices or systems described in this disclosure, e.g., illumination devices 26, 28 of FIGS. 1 and 2, provide light for the operation within a body lumen. The illumination devices may include, but are not limited to, one or more light emitting diodes (LEDs), and/or a fiber optic illumination guide for providing light from a light source, e.g., a laser, a white light source, and the like. The light can be provided as a separate light source from the camera. The light can also be produced by an LED located close to each camera, or an LED located in the handle, in this case the light needs to be transmitted to a location close to the camera with optical fibers. The optical fibers can form a single bundle, multiple bundles, or be incorporated evenly in the circumference of the extension member, inner member and/or outer sheath.

In some example configurations, a lens may be provided at the distal end of an illumination device, e.g., illumination device 28, to focus the illumination on the body lumen or tissue. The illumination device and/or imaging device may include, but is not limited to, an objective lens and fiber optic imaging light guide communicating with a practitioner, a camera, a video display, a cathode ray tube (CRT), a liquid crystal display (LCD), digital light processing (DLP) panel, a plasma display panel (PDP), a light-emitting diode (LED) display, an organic light-emitting diode (OLED) display, a sensor, such as a charge-coupled device (CCD) sensor or a complementary metal oxide semiconductor (CMOS) sensor, and the like for use with a viewing device such as computer displays, video monitors, televisions and the like.

Additionally, in some examples, mirrors or reflective surfaces may be added to the various example configurations described in this disclosure to provide reflective viewing. For example, a mirror located distally may be positioned for the proximal camera to view mirror images therethrough and vice versa. Further, mirrors may be moveable and adjustable to provide a range of viewing from the mirror.

Power and control and video signals to and from first imaging device 12 and illumination device 28 may be provided by a cable assembly contained within proximal portion 13 of articulating tubular member 24. Power and control and video signals to and from second imaging device 16 and illumination device 26 may be provided by a cable assembly contained within the outer sheath 22. Further, circuitry for the imaging devices may be contained within a central handle (not shown) at the proximal end of the delivery device. The circuitry may be powered from a direct current (DC) source, e.g., one or more batteries, or from an alternating current (AC) source. Video signals may be routed out through the central handle for display or processing of the imaging information. In some embodiments, the video signal can be transmitted wirelessly to a receiver located outside the body using known wireless transmission techniques.

FIG. 3 is a schematic view of another example delivery system in accordance with various techniques of this disclosure. FIG. 3 depicts delivery device 30 including inner articulating member 32 extending within outer sheath 36. Outer sheath 36 may be a hollow tubular shaft which covers stent 20 and retains stent 20 in a compressed position until deployment. Inner articulating member 32 extends within the lumen defined by stent 20, and stent 20 extends between inner articulating member 32 and outer sheath 36. Inner articulating member 32 may be a tubular shaft, e.g., solid or hollow, and inner articulating member 32 may have a guidewire extending therethrough (not shown).

Inner articulating member 32 may be a continuous shaft that extends between distal end 40 and a proximal end (not shown). As indicated above, in some example configurations, one or more pull wires may be used to articulate articulating members. For example, in FIG. 3, pull wire 43A may be engaged to a portion of distal tip 42 via an adhesive or fastening device, depicted at 45A, and pull wire 43A may be attached to controls located in a proximal handle (not shown). Distal end 40 includes distal tip 42, which may be hingeably connected at connection point 44 to the remaining shaft of inner articulating member 32, thereby allowing a clinician to articulate distal tip 42 backward onto a distal portion of articulating member 32 by pulling pull wire 43A, for example. FIG. 3 depicts one example of an articulated position.

To articulate distal tip 42 in another direction into another articulated position, a clinician may pull a different pull wire, e.g., pull wire 43B affixed to distal tip 42 at 45B. Pull wires are referred to collectively in this disclosure as “pull wires 43.” In some examples, distal tip 42 can articulate, e.g., rotate about connection point 44, such that imaging device 38 points in a direction that is substantially opposite (about 180°) to the direction that imaging device 38 points in an unarticulated position (shown in solid lines in FIG. 3), as shown in dashes in FIG. 3.

Delivery device 30 may include other pull wires located on other portions of distal tip 42. For example, in some configurations, four pull wires may be provided in order to allow distal tip 42 to articulate in four directions. More or fewer pull wires 43 may be provided. In some examples, pull wires 43 may extend from a proximal end of delivery device 30 (not depicted) to distal end 40 via a channel in the device (not depicted).

In some examples, imaging device 38 is integrally formed from and embedded into distal tip 42, thereby providing a distal view in an unarticulated position and a proximal view in an articulated position. When distal tip 42 is longitudinally aligned with inner articulating member 32 (that is, when distal tip 42 is not in an articulated position), imaging device 38 allows for evaluation of the anatomy prior to stent release. Once in place, a clinician can articulate distal tip 42 using one or more pull wires 43, for example, such that in the articulated position, e.g., hingeably flipped backward, it is adjacent and in parallel alignment with the remaining distal portion 34, as shown in dashes in FIG. 3. In the articulated position, imaging device 38 allows for observation of the distal end of the stent 20 during stent deployment. After stent 20 has been deployed, imaging device 38 can be used to confirm stent placement and re-inspect the anatomy.

Inner articulating member 32 may include imaging device 38, or an imaging device with an illumination device. For example, distal tip 42 may also include an illumination device (not shown). Power and signals to and from imaging device 38 and/or and illumination device may be provided by a cable assembly contained within articulating member 32. Further, support circuitry for the imaging devices may be contained within a central handle (not shown) at the proximal end of the delivery device. The circuitry may be powered from a DC source, e.g., one or more batteries, or an AC source. Video signals may be routed out through the central handle for display or processing of the imaging information. Further, the delivery device may include a pull wire to carry electricity or signals.

Further, in accordance with this disclosure, the delivery devices shown in FIGS. 1-3 may include a distal tip that is opaque or transparent. The distal tip may further include multiple imaging devices therein. Furthermore, the imaging device and illumination device may be located side-by-side or at different locations along the circumference of the articulating member and/or outer sheath. In some example configurations, the articulating member and/or outer sheath can rotate independently from each other for improved visualization. In one example configuration, the distal tip includes colored filters to provide improved viewing of the stent and/or tissue.

FIG. 4 is a schematic view of another example delivery system in accordance with various techniques of this disclosure. FIG. 4 depicts delivery device 50, including inner member 56 extending within outer sheath 52 and stent 20 extending between outer sheath 52 and inner member 56. Specifically, FIG. 4 shows delivery device 50 including inner member 56 extending within outer sheath 52, and extension member 60 extending from the distal end of inner member 56. Outer sheath 52 may be a hollow tubular shaft that covers stent 20 and retains stent 20 in a compressed position until deployment.

FIG. 4 shows stent 20 being released as outer sheath 52 is retracted. Inner member 56 extends within the lumen defined by stent 20 and stent 20 extends between inner member 56 and outer sheath 52. Inner member 56 may be a tubular shaft, e.g., solid or hollow, and may have a guidewire extending therethrough. Inner member 56 may be a continuous shaft extending between distal tip 54 and a proximal end (not shown).

Distal tip 54 includes receiver 58, which may engage connector end 62 of extension member 60. Extension member 60 may be removably attached to inner member 56 by engaging receiver 58 with connector end 62. Receiver 58 and connector end 62 may be engaged using various devices including, but not limited to, a latching device, a snapping device, a threaded device, a magnetic device, and the like. Extension member 60 may be removable to allow for disposal of the remaining delivery device, and extension member 60 may be reuseable by attaching it to another inner member 56 of another delivery device.

Extension member 60 of FIG. 4 may be an elongated articulating tubular shaft 68 extending between distal end 66 and connector end 62. In some examples, extension member 60 may have a diameter equal to or less than the diameter of inner member 56. In one example, elongated articulating tubular shaft 68 may be a solid rod or a hollow tube. In some examples, articulating tubular shaft 68 may define a lumen that allows for the passage of guidewire 72, passage of other material such as injecting contrast medium, or the passage of wires to supply power and video input to and from imaging device 70 and/or the illumination device 64. Elongated articulating tubular shaft 68 can articulate, e.g., retroflex, using a variety of techniques, e.g., shape memory material, pull wires, or electroactive polymers.

It should be noted that a tapered guidewire tip may be added to the distal end of any of the delivery devices described above to improve the ability of the device to traverse strictures. As shown in FIG. 4, guidewire tip 74 is attached to the outer circumference of the distal end of the catheter so as to maintain the forward view of imaging device 70. Tip 74 is tapered away from the end of the catheter to facilitate navigation through strictures. Tip 74 may be constructed of a material such as polyurethane to reduce the potential for tissue perforation. In some examples, the tip 74 may be radiopaque.

Electroactive polymers (EAPs) are characterized by their ability to expand and contract, i.e. volumetric change, in response to electrical stimulation. EAPs can be divided into two categories including electronic EAPs (driven by an electric field) and ionic EAPs (involving mobility or driven by diffusion of ions). Electronic EAPs (electrorestrictive, electrostatic, piezoelectric, ferroelectric) can be induced to change their dimensions by applied electric fields. Examples of materials in this category include ferroelectric polymers (commonly known polyvinylidene fluoride and nylon 11, for example), dielectric EAPs, electrorestrictive polymers such as the electrorestrictive graft elastomers and electro-viscoelastic elastomers, and liquid crystal elastomer composite materials wherein conductive polymers are distributed within their network structure. Ionic EAPs include ionic polymer gels, ionomeric polymer-metal composites, conductive polymers and carbon nanotube composites. Ionic polymer gels are activated by chemical reactions and can become swollen upon a change from an acid to an alkaline environment. Additional information regarding EAPs may be found, for example, in U.S. Pat. No. 7,951,186 to Eidenschink et al., the entire contents of which being incorporated herein by reference.

In example configurations that utilized EAPs, a portion of articulating tubular shaft 68 can be comprised of EAP material. In one example, electrodes may be engaged to portions of the EAP material and voltages can be applied to the electrodes, resulting in electrical fields that cause the EAP material to change shape and articulate in a desired manner.

In some example configurations, the entire elongated articulating tubular shaft 68 can articulate. In one example configuration, only a portion of the elongated articulating tubular shaft 68 can articulate. FIG. 4 shows the distal portion of elongated articulating tubular shaft 68 articulating. Elongated articulating tubular shaft 68 may articulate, e.g., bend backwards, such that imaging device 70 points in a direction that is substantially opposite (about 180°) to the direction that imaging device 70 points in an unarticulated position (shown in solid lines in FIG. 4), as shown in dashes in FIG. 4.

In FIG. 4, angle θ defines an angle between the axis A of inner member 56 and axis B, which is an axis tangential to a point on articulating tubular shaft 68. In order for imaging device 70 to point in a direction that is substantially opposite (about 180°) to the direction that imaging device 70 points in an unarticulated position, angle θ is about 90°. Articulating shaft 68 may articulate at smaller angles. For example, imaging device 70 may point in a direction that is substantially perpendicular (about 90°) to the direction that imaging device 70 points in an unarticulated position (not depicted). In such an example, angle θ is much less than 90°. Other angles in various directions from the line of axis A of inner member 56 are within the scope of this disclosure.

Extension member 60 may include illumination device 64 at distal end 66. Extension member 60 may include one or more imaging devices 70 embedded in the elongated articulating tubular shaft 68. Imaging device 70 may be oriented to provide viewing positions of the distal view or the proximal view. Additionally, imaging device(s) 70 may be rotatable to provide a plurality of viewing positions for the various stages of implanting a stent. It is further contemplated that, in some examples, elongated articulating tubular shaft 68 is transparent to allow imaging device 70 to remain within the perimeter of elongated articulating tubular shaft 68 and not protrude from the surface of elongated articulating tubular shaft 68.

Imaging device 70 and illumination device 64 may be located side-by-side or at different locations along the circumference of the extension member 60. It is further contemplated that extension member 60, inner member 56, and/or outer sheath 52 can rotate independently from each other to allow for better visualization.

The electrical cabling to carry power and signals to and from imaging device 70 and/or illumination device 64 may be contained within inner member 56 and extension member 60. Further, support circuitry for the imaging device(s) may be contained within a central handle (not shown) at the proximal end of the delivery device. The circuitry may be powered from batteries or an AC source. Video signals may be routed out through the central handle for display or processing of the imaging information. In some examples, video signals may be transmitted wirelessly.

Outer tubular members 22, 36, 52 and inner tubular members 24, 32, 56 may be formed of a body compatible material. Desirably, the biocompatible material may be a biocompatible polymer. Examples of suitable biocompatible polymers may include, but are not limited to, polyolefins such as polyethylene (PE), high density polyethylene (HDPE) and polypropylene (PP), polyolefin copolymers and terpolymers, polytetrafluoroethylene (PTFE), polyethylene terephthalate (PET), polyesters, polyamides, polyurethanes, polyurethaneureas, polypropylene and, polycarbonates, polyvinyl acetate, thermoplastic elastomers including polyether-polyester block copolymers and polyamide/polyether/polyesters elastomers, polyvinyl chloride, polystyrene, polyacrylate, polymethacrylate, polyacrylonitrile, polyacrylamide, silicone resins, combinations and copolymers thereof, and the like. Desirably, the biocompatible polymers include polypropylene (PP), polytetrafluoroethylene (PTFE), polyethylene terephthalate (PET), high density polyethylene (HDPE), combinations and copolymers thereof, and the like. Materials for the outer tubular members 22, 36, 52 and/or inner tubular members 24, 32, 56 may be the same or different.

Outer tubular members 22, 36, 52 and/or inner tubular members 24, 32, 56 may also have a surface treatment and/or coating on their inner surface, outer surface or portions thereof. A coating need not be applied to all of outer tubular members 22, 36, 52 and/or inner tubular members 24, 32, 56 and individual members may be coated, uncoated, partially coated, and the like. Useful coating materials may include any suitable biocompatible coating. Non-limiting examples of suitable coatings include polytetrafluoroethylene, silicone, hydrophilic materials, hydrogels, and the like. Useful hydrophilic coating materials may include, but are not limited to, alkylene glycols, alkoxy polyalkylene glycols such as methoxypolyethylene oxide, polyoxyalkylene glycols such as polyethylene oxide, polyethylene oxide/polypropylene oxide copolymers, polyalkylene oxide-modified polydimethylsiloxanes, polyphosphazenes, poly(2-ethyl-2-oxazoline), homopolymers and copolymers of (meth) acrylic acid, poly(acrylic acid), copolymers of maleic anhydride including copolymers of methylvinyl ether and maleic acid, pyrrolidones including poly(vinylpyrrolidone) homopolymers and copolymers of vinyl pyrrolidone, poly(vinylsulfonic acid), acryl amides including poly(N-alkylacrylamide), poly(vinyl alcohol), poly(ethyleneimine), polyamides, poly(carboxylic acids), methyl cellulose, carboxymethylcellulose, hydroxypropyl cellulose, polyvinylsulfonic acid, water soluble nylons, heparin, dextran, modified dextran, hydroxylated chitin, chondroitin sulphate, lecithin, hyaluranon, combinations and copolymers thereof, and the like. Non-limiting examples of suitable hydrogel coatings include polyethylene oxide and its copolymers, polyvinylpyrrolidone and its derivatives; hydroxyethylacrylates or hydroxyethyl(meth)acrylates; polyacrylic acids; polyacrylamides; polyethylene maleic anhydride, combinations and copolymers thereof, and the like. Additional details of suitable coating materials and methods of coating medical devices with the same may be found in U.S. Pat. Nos. 6,447,835 and 6,890,348, the entire contents of each being incorporated herein by reference. Such coatings and/or surface treatment may be disposed on the inside, or a portion thereof, of outer tubular members 22, 36, 52 to facilitate loading and/or deploying of stent 20.

Further, outer tubular members 22, 36, 52 and/or inner tubular members 24, 32, 56 may also include see-through portions to facilitate the delivery of stent 20. Such portions may be transparent, substantially transparent, translucent, substantially translucent and the like. Additional details of delivery devices having such transparent and/or translucent portions may be found in U.S. Patent Application Publication No. 2003/0050686 A1 to Raeder-Devens et al., the entire contents of which being incorporated herein by reference.

While stent 20 may be formed of metals, plastics or other materials, it is preferred that a biocompatible material or construction is employed. Useful biocompatible materials may include, but are not limited to, biocompatible metals, biocompatible alloys, biocompatible polymeric materials, including synthetic biocompatible polymeric materials and bioabsorbable or biodegradable polymeric materials, materials made from or derived from natural sources and combinations thereof. Useful biocompatible metals or alloys may include, but not limited to, nitinol, stainless steel, cobalt-based alloy such as Elgiloy, platinum, gold, titanium, tantalum, niobium, polymeric materials and combinations thereof. Useful synthetic biocompatible polymeric materials include, but are not limited to, polyesters, including polyethylene terephthalate (PET) polyesters, polypropylenes, polyethylenes, polyurethanes, polyolefins, polyvinyls, polymethylacetates, polyamides, naphthalane dicarboxylene derivatives, silks and polytetrafluoroethylenes. The polymeric materials may further include a metallic, a glass, ceramic or carbon constituent or fiber, Useful and nonlimiting examples of bioabsorbable or biodegradable polymeric materials may include poly(L-lactide) (PLLA), poly(D,L-lactide) (PLA), poly(glycolide) (PGA), poly(L-lactide-co-D,L-lactide) (PLLA/PLA), poly(L-lactide-coglycolide) (PLLA/PGA), poly(D,L-lactide-co-glycolide) (PLA/PGA), poly(glycolide-co-trimethylene carbonate) (PGA/PTMC), polydioxanone (PDS), Polycaprolactone (PCL), polyhydroxybutyrate (PHBT), poly(phosphazene) poly(D,L-lactide-co-caprolactone) PLA/PCL), poly(glycolide-co-caprolactone) (PGA/PCL), polyphosphate ester) and the like. Further, stent 20 may include materials made from or derived from natural sources, such as, but not limited to collagen, elastin, glycosaminoglycan, fibronectin and laminin, keratin, alginate, combinations thereof and the like.

In some example configurations, the various articulating tubular members described in this disclosure may be attached to a standard delivery catheter. In one example configuration, the articulating tubular member may be an independent, distinct, and removable mechanism that attaches to or is retrofitted to deployment systems that are known in the art.

In another aspect of the invention, a method for delivering a prosthesis, e.g., stent 20, into a body lumen or a method of use is provided. Device 10, 30, 50 may be used for various applications such as esophageal stenting, colonic stenting, pulmonary stenting, urinary stenting, for various applications for natural orifice transluminal endoscopic surgery (NOTES), biopsy procedures and the like. The method of use includes providing a delivery device 10, 30, 50, the device 10, 30, 50 includes one or more sheaths 22, 36, 52 or stent retaining member to retain the prosthesis, such as a stent, in a compressed state until delivery, and an inner member 24, 32, 56 and at least one imaging device and/or illumination system located on or integrally formed in the inner membrane 24, 32, 56, and a prosthesis or stent 20. The sheath(s) has a proximal end, a distal end, an outer wall and a longitudinal working channel through the sheath defining an inner wall of the sheath and the stent 20 is juxtaposingly disposed to a distal portion of the inner wall and the inner member slidably disposed within the channel. The imaging device is activated to provide imaging during the delivery of the stent and the illumination system is activated to provide illumination within the lumen during the deployment process. The sheath is advanced through the lumen until properly positioned. Once the delivery device 10, 30, 50 is positioned for deployment, the stent 20 may be released from the endoscopic stent delivery device 10, 30, 50 by retracting the elongate sheath to release the stent 20 from the delivery device 10, 30, 50 and/or by advancing the inner member 24, 32, 56 to push the stent 20 out of the delivery device 10, 30, 50. The imaging device provides imaging throughout the deployment of the stent 20 to verify accuracy and placement of the stent. The inner member 24, 32, 56 may be articulated, e.g., moved, bent, tilted, rotated, arched, via shape memory material, pull wires, and EAP to position the imaging device and/or illumination device located thereon for better visual imaging of the lumen, stent, deployment process and verification of proper positioning. The step of providing the endoscopic stent delivery device 10, 30, 50 may further include a step of loading the stent 20 within the distal portion of the inner wall of the endoscope 10, 30, 50. The method may further include radially compressing the stent 20 prior to loading the stent 20 within the distal portion of the inner wall of the endoscope 10, 30, 50.

Additionally, the method of use includes selecting the proper prosthesis, e.g., stent, according to the patient anatomy and disease progression; loading the desired prosthesis into the delivery device 10, 30, 50 or selecting a pre-loaded delivery device 10, 30, 50 including the proper prosthesis; connecting the delivery device to external equipment to supply power and necessary external elements to the device; introducing the device through the desired orifice and extending the device through a lumen to the location for deployment; confirming proper positioning by direct visual confirmation and exploring the lumen and/or stricture to ensure proper placement of prosthesis, e.g., the esophago-gastroenoscopy (EGD) is performed by the device; measuring the stricture and recording the measurements; advancing a guidewire into the invention through the stricture if needed; moving the inner articulate member to provide direct visualization of the lumen, stent, deployment process, verification of proper positioning; deploying the prosthesis by pulling back on the sheath while the physician watched the deployment under direct visualization by the cameras; ensuring proper placement of the prosthesis by direct visualization once the prosthesis has been deployed; removing the device from the lumen. Additionally, it is contemplated that the imaging device and/or illumination system may be attached to the device integrally formed on the inner member prior to introducing the device with the lumen. Further, it is contemplated that the inner member may be attached or retrofitted onto a delivery device prior to introducing the device onto the lumen.

While the invention has been described by reference to certain preferred embodiments, it should be understood that numerous changes could be made within the spirit and scope of the inventive concept described. Accordingly, it is intended that the invention not be limited to the disclosed embodiments, but that it have the full scope permitted by the language of the following claims.

1. A delivery device comprising:
at least one sheath removably covering a stent therein, said at least one sheath comprising a distal end, a proximal end, an outer surface and a working channel extending between said distal end and said proximal end, said working channel defining an inner wall;
said stent defining a stent lumen, said stent extending in a compressed state within said working channel;
an inner tubular member slidably disposed within said stent lumen, said inner tubular member comprising an elongated inner shaft with a distal articulating portion extending therefrom; and
at least one imaging device integrally formed in said distal articulating portion.

2. The delivery device of claim 1, wherein said distal articulating portion further comprises an illumination device integrated into said elongated inner shaft.

3. The delivery device of claim 1, wherein said distal articulating portion is segmented to provide articulation of said distal articulating portion.

4. The delivery device of claim 2, wherein said at least one sheath further comprises an imaging device integrated into and embedded into said at least one sheath and integrally formed from said outer surface.

5. The delivery device of claim 1, wherein said distal articulating portion comprises a distal tip, said at least one imaging device is integrally formed and embedded into said distal tip.

6. The delivery device of claim 4, wherein said distal articulating portion comprises a distal tip, said at least one imaging device is integrally formed and embedded into said distal tip.

7. The delivery device of claim 6, wherein said distal tip comprises two of said at least one imaging devices, and wherein the two imaging devices are located on either side of said at least one imaging device.

8. The delivery device of claim 4, wherein said distal articulating portion is segmented to provide articulation of said distal articulating portion.

9. The delivery device of claim 1, wherein said distal articulating portion is removably attached to said inner elongate shaft.

10. The delivery device of claim 9, wherein said distal articulating portion comprises a first proximal end, a first distal end, and an elongated articulating shaft extending therebetween, said first distal end includes a guidewire extending from said first distal end.

11. The delivery device of claim 9, wherein said distal articulating portion comprises a first proximal end, a first distal end, and an elongated articulating shaft extending therebetween, wherein said elongated articulating shaft has a diameter equal to or less than a diameter of said inner elongate shaft.

12. The delivery device of claim 11, wherein said first distal end comprises a guidewire extending from said first distal end.

13. The delivery device of claim 12, further including illumination device integrally formed and embedded in said first distal end.

14. The delivery device of claim 9, wherein said distal articulating portion is segmented to provide articulation of said distal articulating portion.

15. The delivery device of claim 1, wherein said distal articulating portion is hingeably attached to said elongated inner shaft.

16. The delivery device of claim 15, further including an illumination device integrated and embedded into said distal articulating portion.

17. The delivery device of claim 16, wherein said distal articulating portion further includes a first distal end and a first proximal end, said imaging device is integrally formed and embedded into said first distal end.

18. A method for intraluminally positioning a prosthesis comprising:
providing a delivery device comprising at least one sheath removably covering a prosthesis therein, said at least one sheath comprising a distal end, a proximal end, an outer surface and a working channel extending between said distal end and said proximal end, said working channel defining an inner wall, said prosthesis extending in a compressed state within said working channel, an inner tubular member slidably disposed within said prosthesis, said inner tubular member comprises an elongated inner shaft with a distal articulating portion extending therefrom, and at least one imaging device integrally formed in said distal articulating position;
activating said at least one imaging device to provide images during positioning of said prosthesis;
positioning said delivery device within a body lumen; and
slidably retracting said at least one sheath relative to the inner tubular member to uncover said prosthesis and allow said prosthesis to radially expand against a wall of body lumen, wherein said articulating position is bent back upon itself to allow said at least one imaging device to be positioned for visual inspection of deployment of the prosthesis while slidably retracting said at least one sheath.
